(19) United States
(12) Patent Application Publication
(10) Pub. No.: US 2014/0308244 A1
(43) Pub. Date: Oct. 16, 2014

(54) Combination Therapy for Treatment of Inflammatory Demyelinating Disease

(71) Applicants: Lawrence Steinman, Stanford, CA (US); Peggy Pui-Kay Ho, Cupertino, CA (US)

(72) Inventors: Lawrence Steinman, Stanford, CA (US); Peggy Pui-Kay Ho, Cupertino, CA (US)

(73) Assignee: The Board of Trustees of the Leland Stanford Junior University, Palo Alto, CA (US)

(21) Appl. No.: 14/236,877

(22) PCT Filed: Aug. 7, 2012
(86) PCT No.: PCT/US12/49844

§ 371 Date: Jul. 2, 2014

Related U.S. Application Data
(60) Provisional application No. 61/521,273, filed on Aug. 8, 2011.

(51) Int. Cl. A61K 31/40; A61K 31/137; A61K 31/225; A61K 38/21; A61K 31/714

(52) U.S. Cl. CPC A61K 31/40; A61K 38/215; A61K 31/714; A61K 31/225; A61K 31/137
USPC 424/85.6; 514/423; 514/52

(57) ABSTRACT

Provided herein include combination therapies for the treatment of neurological inflammatory diseases, such as, for example, demyelinating autoimmune diseases, such as multiple sclerosis and neuromyelitis optica, etc. In various aspects and embodiments, the methods may include administering to a patient an effective dose of an inhibitor of an angiotensin-converting enzyme (ACE) in combination with one or more second compounds, In one aspect, provided is a method of treating a demyelinating autoimmune disease (such as multiple sclerosis) that includes administering to a patient an effective dose of an inhibitor of an angiotensin-converting enzyme (ACE) in combination with one or more compounds selected from the group consisting of a cytokine, a vitamin B, dimethyl fumarate (DMF, also referred to as BG-12) and fingolimod (Gilenya).

### BACKGROUND

Platten et al. (2009) PNAS 106(35):14948-14953 discuss the involvement of ACE-inhibitors in autoimmune disease.

### SUMMARY OF THE INVENTION

Provided herein include combination therapies for the treatment of neurological inflammatory diseases, such as, for example, demyelinating autoimmune diseases, such as multiple sclerosis and neuromyelitis optica, etc.

In various aspects and embodiments, the methods may include administering to a patient an effective dose of an inhibitor of an angiotensin-converting enzyme (ACE) in combination with one or more second compounds, In one aspect, provided is a method of treating a demyelinating autoimmune disease (such as multiple sclerosis) that includes administering to a patient an effective dose of an inhibitor of an angiotensin-converting enzyme (ACE) in combination with one or more compounds selected from the group consisting of a cytokine, a vitamin B, dimethyl fumarate (DMF, also referred to as BG-12) and fingolimod (Gilenya). In some embodiments the ACE inhibitor is lisinopril. In some embodiments the cytokine is β-interferon. In some embodiments the vitamin B is vitamin B12. The effective dose of each drug in a combination therapy may be lower than the effective dose of the same drug in a monotherapy. In some embodiments the combined therapies are administered concurrently. In some embodiments the two therapies are phased, for example where one compound is initially provided as a single agent, e.g. as maintenance, and where the second compound is administered during a relapse, for example at or following the initiation of a relapse, at the peak of relapse, etc.

In one aspect, provided is a method of treating a demyelinating autoimmune disease (such as multiple sclerosis) that includes administering to a patient an effective dose of an inhibitor of an angiotensin-converting enzyme (ACE) in combination with a cytokine. In some embodiments the ACE inhibitor is lisinopril. In some embodiments the cytokine is β-interferon. In some embodiments the combined therapies are administered concurrently. In some embodiments the two therapies are phased, for example where the cytokine is initially provided as a single agent, e.g. as maintenance, and where the ACE inhibitor is administered during a relapse, for example at or following the initiation of a relapse, at the peak of relapse, etc. Alternatively the ACE inhibitor is initially provided as a single agent, e.g. as maintenance, and the cytokine is administered during a relapse, for example at or following the initiation of a relapse, at the peak of relapse, etc.

In one aspect, provided is a package (for example a box, a bottle or a bottle and box) that includes an ACE inhibitor and a package insert or label that indicates that the ACE inhibitor is to be administered in combination with a second compound to a patient for the treatment of a demyelinating autoimmune disease (such as multiple sclerosis). In some embodiments the second compound is one or more compounds selected from the group consisting of a cytokine, a vitamin B, dimethyl fumarate (DMF, also referred to as BG-12) and fingolimod (Gilenya). In certain embodiments, the package includes an ACE inhibitor, one or more second compounds selected from the group consisting of a cytokine, a vitamin B, dimethyl fumarate (DMF, also referred to as BG-12) and fingolimod (Gilenya) and a package insert or label that indicates that the ACE inhibitor is to be administered in combination with the second compound to a patient for the treatment of a demyelinating autoimmune disease (such as multiple sclerosis). In some embodiments the ACE inhibitor is lisinopril. In some embodiments the cytokine is β-interferon. In some embodiments the vitamin B is vitamin B12.

In one aspect, provided is a composition for oral administration (e.g., a pill, capsule, tablet, syrup, emulsion, liquid, elixir and the like) that includes an ACE inhibitor and one or more compounds selected from the group consisting of a cytokine, a vitamin B, dimethyl fumarate (DMF, also referred to as BG-12) and fingolimod (Gilenya). In some embodiments the ACE inhibitor is lisinopril. In some embodiments the cytokine is β-interferon. In some embodiments the vitamin B is vitamin B12.

In some embodiments of the invention, the patient is analyzed for responsiveness to cytokine therapy, where the selection of cytokine in the combination therapy is based on such analysis. The efficacy of immunomodulatory treatments of inflammatory demyelinating diseases of the central nervous system, e.g. multiple sclerosis, neuromyelitis optica, EAE, etc., depends on whether a patient has a predominantly TH1-type disease subtype, or a predominantly TH17-type disease subtype. Patients can be classified into subtypes by determining the levels of markers, including IL-17; endogenous β-interferon, IL-23, PDGFBB, sFAS ligand, M-CSF, MIP1α, TNF-β, IFNα, IL-1RA, MCP-1, IL-2, IL-6, IL-8, FGFβ, IL-7, TGF-β, IFNβ, IL-13, IL-17F, EOTAXIN, IL-1α, MCP-3, LIF, NGF, RANTES, IL-5, MIP1b, IL-12p70, and HGF, etc. Cytokines such as β-interferon may be administered to individuals having a predominantly TH1-type disease subtype, as disclosed in co-pending U.S. application Ser. No. 13/026,173. herein specifically incorporated by reference.

### BRIEF DESCRIPTION OF THE DRAWINGS

FIG. 1 is a graph of disease scores for EAE, when Rebif is administered 1 day after immunization for EAE at 1 μg/mouse, and given intraperitoneally every other day for 5 doses (pro-treatment), and Lisinopril is administered daily, orally, at the peak of disease (treatment).

FIG. 2 is a graph of disease scores for EAE, when combination treatment of Rebif and Lisinopril is initiated at the peak of disease.

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present inventions have been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Multiple sclerosis (MS) is an inflammatory and degenerative disease of the central nervous system (CNS) with diverse clinical presentations and heterogeneous histopathological features. In MS, myelin reactive T cells enter into the brain and spinal cord and mediate destruction of the myelin sheath surrounding neurons resulting in progressive motor dysfunction and eventual paralysis. Current treatment strategies include switching the pro-inflammatory Th1 T cell phenotype to an anti-inflammatory Th2 response, preventing encephalitogenic T cells from extravasating into the brain, inducing T cell tolerance, anergy or apoptosis, and repairing or replacing damaged CNS cells, such as neurons and oligodendrocytes.

Goals for therapy include shortening acute exacerbations, decreasing frequency of exacerbations, and relieving symptoms; maintaining the patient's ability to walk is particularly important. Acute exacerbations may be treated with brief courses of corticosteroids. However, although they may shorten acute attacks and perhaps slow progression, corticosteroids have not been shown to affect long-term outcome.

Immunomodulatory therapy decreases frequency of acute exacerbations and delays eventual disability. Immunomodulatory drugs include interferons (IFNs), such as IFN-β1b and IFN-β1a. Glatiramer acetate may also be used. Other potential therapies include the immunosuppressant methotrexate and Natalizumab, an anti-α₄ integrin antibody that inhibits passage of leukocytes across the blood-brain barrier. Immunosuppressants such as mycophenolate and cyclophosphamide have been used for more severe, progressive MS but are controversial.

There is a long-standing interest in manipulating cells of the immune system to achieve control of autoimmune disease. While targeted antigen-specific therapy remains of great interest, there has also been considerable development of polyclonal, or non-antigen specific therapies. In addition to general immunosuppression, e.g. through the use of agents such as hydrocortisone, many therapies are now being brought to the clinic that provide for a more selective modification of the immune system, such as modulation of cytokines.

Improvement in the use of disease-modifying therapies in autoimmune conditions is of great clinical interest. In certain aspects and embodiments the present methods and compositions address this need.

The subject methods may be used for prophylactic or therapeutic purposes. As used herein, the term “treating” is used to refer to both prevention of relapses, and treatment of pre-existing conditions. For example, the prevention of autoimmune disease may be accomplished by administration of the agent prior to development of a relapse. The treatment of ongoing disease, where the treatment stabilizes or improves the clinical symptoms of the patient, is of particular interest.

“Diagnosis” as used herein generally includes determination of a subject's susceptibility to a disease or disorder, determination as to whether a subject is presently affected by a disease or disorder, prognosis of a subject affected by a disease or disorder (e.g., identification of disease states, stages of MS, or responsiveness of MS to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, for example humans, non-human primate, mouse, rat, guinea pig, rabbit, etc.

“Inhibiting” the onset of a disorder shall mean either lessening the likelihood of the disorder's onset, or preventing the onset of the disorder entirely. Reducing the severity of a relapse shall mean that the clinical indicia associated with a relapse are less severe in the presence of the combination therapy than in an untreated disease, or in the presence of a single agent treatment with the cytokine or ACE inhibitor. As used herein, onset may refer to a relapse in a patient that has ongoing relapsing remitting disease. The methods of the invention are specifically applied to patients that have been diagnosed with an autoimmune disease. Treatment is aimed at the treatment or reducing severity of relapses, which are an exacerbation of a pre-existing condition.

“Inhibiting” the expression of a gene in a cell shall mean either lessening the degree to which the gene is expressed, or preventing such expression entirely.

Inflammatory demyelinating disease. The term “inflammatory” response is the development of a humoral (antibody mediated) and/or a cellular (mediated by antigen-specific T cells or their secretion products) response. Inflammatory demyelinating diseases of the central nervous system are of particular interest and include, without limitation, multiple sclerosis (MS), neuromyelitis optica (NO), and experimental acquired encephalitis (EAE). Demyelinating inflammatory diseases of the peripheral nervous system include Guillain-Barre syndrome (GBS) with its subtypes acute inflammatory demyelinating polyradiculoneuropathy, acute motor axonal neuropathy, acute motor and sensory axonal neuropathy, Miller Fisher syndrome, and acute pandysautonomia; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy.

Multiple sclerosis is characterized by various symptoms and signs of CNS dysfunction, with remissions and recurring exacerbations. Classifications of interest for analysis by the methods of the invention include relapsing remitting MS (RRMS), primary progressive MS (PPMS) and secondary progressive MS (SPMS). The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat can accentuate symptoms and signs.

The course is highly varied, unpredictable, and, in most patients, remittent. At first, months or years of remission can separate episodes, especially when the disease begins with retrobulbar optic neuritis. However, some patients have frequent attacks and are rapidly incapacitated; for a few the course can be rapidly progressive (primary progressive MS, PPMS), or secondary progressive multiple sclerosis (SPMS). Relapsing remitting MS (RR MS) is characterized clinically by relapses and remissions that occur over months to years, with partial or full recovery of neurological deficits between attacks. Such patients manifest approximately 1 attack, or relapse, per year. Over 10 to 20 years, approximately 50% of RR MS patients develop secondary progressive MS (SP MS) which is characterized by incomplete recovery between attacks and accumulation of neurologic deficits resulting in increasing disability.

Diagnosis is usually indirect, by deduction from clinical, radiographic (brain plaques on magnetic resonance [MR] scan), and to a lesser extent laboratory (oligoclonal bands on CSF analysis) features. Typical cases can usually be diagnosed confidently on clinical grounds. The diagnosis can be suspected after a first attack. Later, a history of remissions and exacerbations and clinical evidence of CNS lesions disseminated in more than one area are highly suggestive.

MRI, the most sensitive diagnostic imaging technique, can show plaques. It can also detect treatable nondemyelinating lesions at the junction of the spinal cord and medulla (eg, subarachnoid cyst, foramen magnum tumors) that occasionally cause a variable and fluctuating spectrum of motor and sensory symptoms, mimicking MS. Gadolinium-contrast enhancement can distinguish areas of active inflammation from older brain plaques. MS lesions can also be visible on contrast-enhanced CT scans; sensitivity can be increased by giving twice the iodine dose and delaying scanning (double-dose delayed CT scan).

Neuromyelitis optica (NMO), or Devic's disease, is an autoimmune, inflammatory disorder of the optic nerves and spinal cord. Although inflammation can affect the brain, the disorder is distinct from multiple sclerosis, having a different pattern of response to therapy, possibly a different pattern of autoantigens and involvement of different lymphocyte subsets.

The main symptoms of Devic's disease are loss of vision and spinal cord function. As for other etiologies of optic neuritis, the visual impairment usually manifests as decreased visual acuity, although visual field defects, or loss of color vision can occur in isolation or prior to formal loss of acuity. Spinal cord dysfunction can lead to muscle weakness, reduced sensation, or loss of bladder and bowel control. The damage in the spinal cord can range from inflammatory demyelination to necrotic damage of the white and grey matter. The inflammatory lesions in Devic's disease have been classified as type II lesions (complement mediated demyelinization), but they differ from MS pattern II lesions in their prominent perivascular distribution. Therefore, the pattern of inflammation is often quite distinct from that seen in MS.

Attacks are conventionally treated with short courses of high dosage intravenous corticosteroids such as methylprednisolone IV. When attacks progress or do not respond to corticosteroid treatment, plasmapheresis can be used. Commonly used immunosuppressant treatments include azathioprine (Imuran) plus prednisone, mycophenolate mofetil plus prednisone, Rituximab, Mitoxantrone, intravenous immunoglobulin (IVIG), and cyclophosphamide.

The disease can be monophasic, i.e. a single episode with permanent remission. However, at least 85% of patients have a relapsing form of the disease with repeated attacks of transverse myelitis and/or optic neuritis. In patients with the monophasic form the transverse myelitis and optic neuritis occur simultaneously or within days of each other. Patients with the relapsing form are more likely to have weeks or months between the initial attacks and to have better motor recovery after the initial transverse myelitis event. Relapses usually occur early with about 55% of patients having a relapse in the first year and 90% in the first 5 years. Unlike MS, Devic's disease rarely has a secondary progressive phase in which patients have increasing neurologic decline between attacks without remission. Instead, disabilities arise from the acute attacks.

Cytokines. As used herein, the term refers to any one of the cell-signaling protein molecules that are secreted by cells of the immune system that have immunomodulating activity, including particularly interleukins and interferons. Of particular interest is β-interferon.

Interleukin-17 (IL-17) refers to a group of cytokines called the IL-17 family. IL-17 shows high homology to viral IL-17 encoded by an open reading frame of the T lymphotropic rhadinovirus Herpesvirus saimiri. To elicit its functions, IL-17 binds to a type I cell surface receptor called IL-17R of which there are at least three variants IL17RA, IL17RB, and IL17RC. Inhibitors of IL-17 include, without limitation, antibodies specific for the cytokine and/or its receptor. For example, AIN457, is a fully human antibody to interleukin-17A. “IL-17F” as used herein refers to IL-17F monomers or multimers containing at least one IL-17F monomer. “IL-17A” as used herein refers to IL-17A monomers or multimers containing at least one IL-17A monomer. “IL-17” as used herein can refer to either IL-17F or IL-17A. Increased levels of II-17F and baseline levels of IL-17A are indicative of NMO or β-IFN non-responsive MS. In some embodiments of the invention, a patient is initially selected for β-IFN responsiveness, i.e. a patient in which levels of IL-17F are not increased relative to a control patient population.

Interleukin-7. For sequence information of the human protein, see Goodwin et al. (1989) Proc Natl Acad Sci USA. 86:302-306, herein specifically incorporated by reference. The gene for human IL-7 is located on chromosome 8q12-13, spans 6 exons, and has open-reading frame of 534 base pairs (177 amino acids), including a 25-amino acid signal peptide. IL-7 is a member of the family of cytokines that signal through the common cytokine gamma chain (γc). IL-7 also uses a second component, the IL-7 receptor alpha chain (IL-7Rα) (CD127). Signaling through the IL-7R requires both IL-7Rα and the γc component. Those patients responsive to TH1-therapy, such as administration of β-IFN, can be characterized has having a level of IL-7 that is significantly higher than a non-diseased individual, while those patients non-responsive to such therapy have a level of IL-7 not significantly different than a non-diseased individual. In some embodiments of the invention, a patient is initially selected for β-IFN responsiveness, i.e. a patient in which levels of IL-7 are increased relative to a control patient population.

Interferon beta is a drug in the interferon family used to treat multiple sclerosis (MS). IFN-β1a is produced by mammalian cells while Interferon beta-1b is produced in modified E. coli. Interferons have been shown to have about a 18-38% reduction in the rate of MS relapses, and to slow the progression of disability in MS patients. Commercially available products include Avonex (Biogen Idec); Rebif (EMD Serono); and CinnoVex (CinnaGen). Closely related is Interferon beta-1b, which is marketed in the US as Betaseron, or Extavia.

Various formulations and dosages are conventionally utilized in the treatment of MS patients with IFN-β, which doses may be utilized in the combination treatments of the present invention, or may be utilized at a lower dose, e.g. 90% of the conventional dose, 80% of the conventional dose, 70% of the conventional dose, 60% of the conventional dose, 50% of the conventional dose, or less.

Avonex is sold in two formulations, a lyophilized powder requiring reconstitution and a pre-mixed liquid syringe kit; it is usually administered once per week via intramuscular injection at a dose of 30 μg. Rebif is administered via subcutaneous injection three times per week at a dose of 22 μg or 44 μg. Interferon beta-1b is usually administered at 250 μg on alternate days.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular (mediated by antigen-specific T cells or their secretion products) response. An “immunogen” is capable of inducing an immunological response against itself on administration to a mammal or due to autoimmune disease.

The terms “biomarker,” “biomarkers,” “marker” or “markers” refer to, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. Markers can also include mutated proteins, mutated nucleic acids, variations in copy numbers and/or transcript variants. Markers also encompass non-blood borne factors and non-analyte physiological markers of health status, and/or other factors or markers not measured from samples (e.g., biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences. Markers can include PDGFBB, sFAS ligand, M-CSF, MIP1a, TNF-B, IFNa, IL-1RA, MCP-1, IL-2, IL-6, IL-8, FGFb, IL-7, TGF-b, IFNb, IL-13, IL-17F, EOTAXIN, IL-1a, MCP-3, LIF, NGF, RANTES, IL-5, MIP1b, IL-12p70, and/or HGF.

A “subject” or “patient” in the context of the present teachings is generally a mammal. Mammals other than humans can be advantageously used as subjects that represent animal models of inflammation. A subject can be male or female.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis to, e.g., determine whether a subject is a responder or a non-responder to a therapy (e.g., an IFN treatment as described herein).

ACE inhibitors. ACE inhibitors or angiotensin-converting enzyme inhibitors are a group of drugs conventionally used for the treatment of hypertension (high blood pressure) and congestive heart failure. They inhibit angiotensin-converting enzyme (ACE), a component of the blood pressure-regulating renin-angiotensin system. ACE inhibitors block the conversion of angiotensin I to angiotensin II.

ACE inhibitors can be divided into three groups based on their molecular structure. Sulfhydryl-containing agents include Captopril (trade name Capoten), and Zofenopril. Dicarboxylate-containing agents include Enalapril (Vasotec/Renitec), Ramipril (Altace/Tritace/Ramace/Ramiwin), Quinapril (Accupril), Perindopril (Coversyl/Aceon), Lisinopril (Listril/Lopril/Novatec/Prinivil/Zestril), Benazepril (Lotensin), Imidapril (Tanatril), and Zofenopril (Zofecard). Phosphonate-containing agents include Fosinopril (Monopril). The lactotripeptides Val-Pro-Pro and Ile-Pro-Pro have been shown to have ACE-inhibiting functions.

The ACE inhibitors have different strengths with different starting dosages. Dosage may be adjusted according to the clinical response. Conventional daily dosages include Benazepril 10 mg to 80 mg; Captopril 50 mg to 450 mg; Enalapril 5 mg to 40 mg; Fosinopril 10 mg to 80 mg; Lisinopril 10 mg to 80 mg; Moexipril 7.5 mg to 30 mg; Perindopril 4 mg to 16 mg; Quinapril 10 mg to 80 mg; Ramipril 2.5 mg to 20 mg; Trandolapril 2 mg to 8 mg.

In some embodiments the ACE inhibitor is lisinopril, administered orally. Administration may be daily, twice daily, etc., with a total daily dose of at least about 10 mg, at least about 25 mg, at least about 50 mg, at least about 75 mg, at least about 100 mg, and usually not more than about 500 mg.

In some embodiments, dimethyl fumarate (DMF, BG12, BG00012, Panaclar) is a second compound in the methods and compositions described herein. In certain embodiments BG12 is administered orally (for example in an amount of about 240 mg per dose) twice or three times daily.

In some embodiments, fingolimod (Gilenya) is a second compound in the methods and compositions described herein. In certain embodiments BG12 is administered orally at a dose of about 0.25 mg/day; or about 0.5 mg/day; or about 0.75 mg/day; or about 1 mg/day; or about 1.25 mg/day; or about 1.5 mg/day.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

### METHODS

The present disclosure provides methods for treating neurological inflammatory diseases, which may be a demyelinating autoimmune disease, such as multiple sclerosis. The methods may include administering to the subject an effective amount of an agent that inhibits angiotensin-converting enzyme (ACE) in combination with a second compound such as one or more compounds selected from the group consisting of a cytokine, a vitamin B, dimethyl fumarate (DMF, also referred to as BG-12) and fingolimod (Gilenya). In some embodiments the cytokine is IFN-β. In some embodiments the ACE inhibitor is lisinopril. In some embodiments the vitamin B is vitamin B12.

In some embodiments the combined therapies are administered concurrently, where the administered dose of any one of the compounds may be a conventional dose, or less than a conventional dose. In some embodiments the two therapies are phased, for example where one compound is initially provided as a single agent, e.g. as maintenance, and where the second compound is administered during a relapse, for example at or following the initiation of a relapse, at the peak of relapse, etc.

In some embodiments, the patient is analyzed for responsiveness to cytokine therapy, where the selection of cytokine in the combination therapy is based on such analysis.

In various aspects and embodiments of the methods and compositions described herein, administering the therapeutic compositions can be effected or performed using any of the various methods and delivery systems known to those skilled in the art. The administering can be performed, for example, intravenously, orally, via implant, transmucosally, transdermally, intramuscularly, intrathecally, and subcutaneously. The delivery systems employ a number of routinely used pharmaceutical carriers.

In various embodiments the two compounds of the combination therapy can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method. Usually the administration is by conventional route.

The compositions can be administered in a single dose, or in multiple doses, usually multiple doses over a period of time, e.g. daily, every-other day, weekly, semi-weekly, monthly etc. for a period of time sufficient to reduce severity of the inflammatory disease, which can comprise 1, 2, 3, 4, 6, 10, or more doses.

Determining a therapeutically or prophylactically effective amount of a combination of agents according to the present methods can be done based on animal data using routine computational methods. The effective dose will depend at least in part on the route of administration.

Treating, treatment, or therapy of a disease or disorder shall mean lessening the severity of adverse clinical symptoms by administration of an ACE inhibitor composition. As used herein, ameliorating a disease and treating a disease are equivalent.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### EXAMPLES

### Combination Therapy of Angiotensin-Converting Enzyme Inhibitor, Lisinopril, and Beta-Interferon for the Treatment of Multiple Sclerosis

It has been previously reported that treatment with the angiotensin-converting enzyme (ACE) inhibitor, Lisinopril, was effective in treating the experimental autoimmune encephalomyelitis (EAE), the mouse model for multiple sclerosis (MS), (Platten et al, 2009, PNAS 106:14948). Angiotensin type 1 receptors were found to be induced in myelin-specific CD4+ T cells and monocytes during autoimmune neuroinflammation. The method of action was through the suppression of autoreactive Th1 and Th17 cells, the promotion of antigen-specific CD4+FoxP3+ regulatory T cells, and the inhibition of the canonical NF-kB1 transcription factor complex and activation of the alternative NF-kB2 pathway.

IFN-β therapy is approved as four different forms, Avonex, Betaseron, Extavia, and Rebif, for the treatment of MS. Lisinopril is a generic drug that is approved in the U.S. to treat hypertension and congestive heart failure, and to improve survival after a heart attack. A synergistic effect may be found for a combination of Lisinopril and IFN-β in treating relapsing EAE and MS.

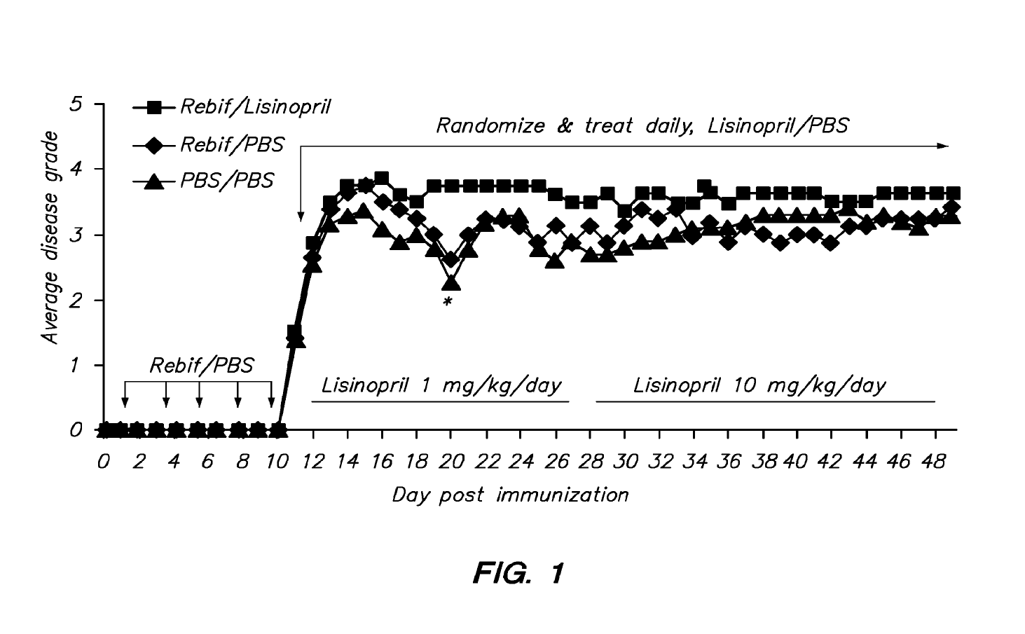

In vivo data show that if Rebif is administered 1 day after immunization for EAE at 1 μg/mouse, and given intraperitoneally every other day for 5 doses (pro-treatment), and Lisinopril is administered daily, orally, at the peak of disease (treatment), the average relapse rate over 50 days is 1.25 versus Rebif pro-treatment combined with PBS (relapse rate 2.0), see Table 1. This data was compelling, given that the overall disease scores were unremarkable, compared to the control PBS group (FIG. 1).

TABLE 1

| Treatment | Average Relapse Rate |
|---|---|
| Rebif/Lisinopril | 1.25 |
| Rebif/PBS | 2.00 |
| PBS/PBS | 1.38 |

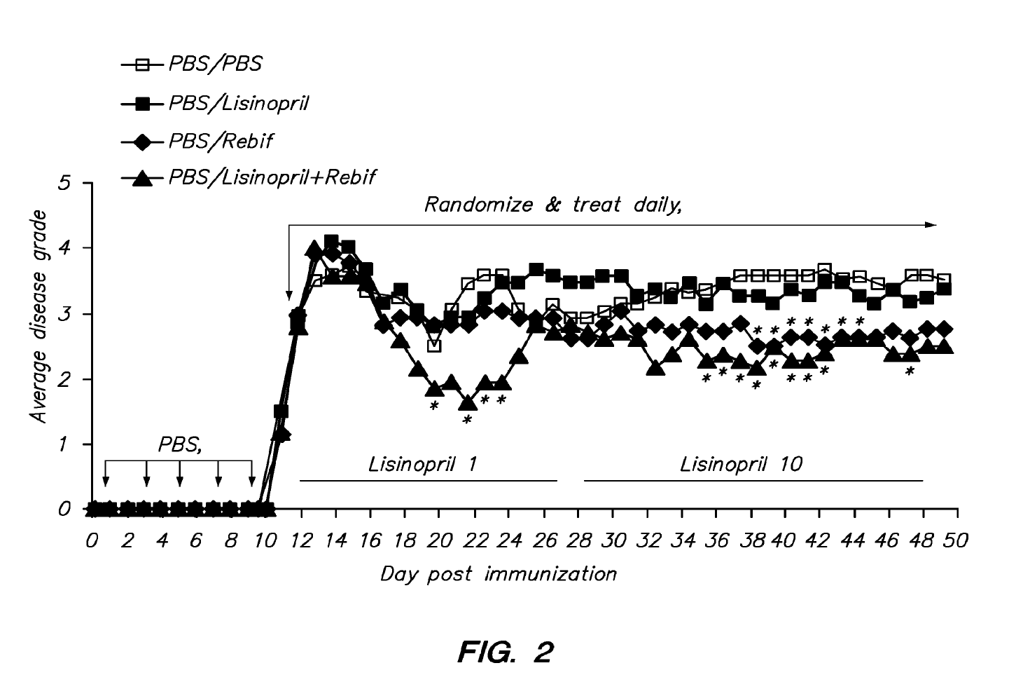

When combination treatment of Rebif and Lisinopril is initiated at the peak of disease (treatment), the overall disease score is statistically lower than control PBS treatment. In this instance, Rebif was given daily (0.25 μg/mouse, intraperitoneally), and Lisinopril was given daily (1 mg/kg/day, orally). Moreover, combination therapy is more clinically effective than treatment with Rebif alone (daily, 0.25 μg/mouse, intraperitoneally) or Lisinopril alone (1 mg/kg/day, orally). See FIG. 2.

The average relapse rate of combination therapy (1.0) is decreased from PBS control treatment (1.38). Relapse rate for Lisinopril alone (0.7) and Rebif alone (1.0). See Table 2.

TABLE 2

| Treatment | Average Relapse Rate |
|---|---|
| PBS/PBS | 1.38 |
| PBS/Lisinopril | 0.70 |
| PBS/Rebif | 1.00 |
| PBS/Lisinopril + Rebif | 1.00 |

1. (canceled)

2. A method for treating an inflammatory demyelinating disease in a patient, the method comprising: administering to said patient a therapeutically effective dose of a combination of agents, wherein a first agent is an inhibitor of angiotensin-converting enzyme (ACE) activity and a second agent is one or more compounds selected from the group consisting of a cytokine, a vitamin B, dimethyl fumarate (DMF, also referred to as BG-12) and fingolimod (Gilenya).

3. (canceled)

4. A composition comprising a package comprising an ACE inhibitor and a package insert or label that indicates that the ACE inhibitor is to be administered in combination with a second compound to a patient for the treatment of a demyelinating autoimmune disease; wherein said second compound is one or more compounds selected from the group consisting of a cytokine, a vitamin B, dimethyl fumarate (DMF, also referred to as BG-12) and fingolimod (Gilenya).

5. A composition for oral administration, comprising an ACE inhibitor and one or more compounds selected from the group consisting of a cytokine, a vitamin B, dimethyl fumarate (DMF, also referred to as BG-12) and fingolimod (Gilenya).

6. The method of claim 2, wherein said disease is multiple sclerosis.

7. The method of claim 2, wherein said inhibitor of ACE activity is lisinopril.

8. The method of claim 2, wherein the cytokine, if present is IFN-β.

9. The method of claim 2, wherein the cytokine if present is IFN-β and the inhibitor of ACE activity is lisinopril.

10. The method of claim 2, wherein said second agent or compound comprises a vitamin B.

11. The method of claim 2, wherein said second agent or compound comprises vitamin B12.

12. The method of claim 2, wherein said second agent or compound comprises dimethyl fumarate.

13. The method of claim 2, wherein said second agent or compound comprises fingolimod (Gilenya).

14. The method of claim 2, wherein the combination provides for a synergistic response.

15. The method of claim 8, wherein the IFN-β is administered at less than a conventional dose.

16. The method of claim 2, wherein the agents are administered concurrently.

17. The method of claim 2, wherein the agents are phased in administration.

18. The method of claim 8, wherein the IFN-β is administered at a maintenance dose as a single agent, and the ACE inhibitor is administered as a second agent during a relapse.

19. The method of claim 2, wherein the patient is patient is analyzed for responsiveness to cytokine therapy, and where the selection of cytokine in the combination therapy is based on such analysis.
